(19) United States
(12) Patent Application Publication
(10) Pub. No.: US 2003/0022932 A1
(43) Pub. Date: Jan. 30, 2003

(54) Methods for regulating bacteria

(76) Inventors: Michael G. Surette, Calgary (CA); Jeffrey L. Stein, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/151,189

(22) Filed: May 17, 2002

Related U.S. Application Data
(60) Provisional application No. 60/292,543, filed on May 21, 2001.

(51) Int. Cl. A61K 31/365; G01N 33/554; G01N 33/569; A61K 31/12

(52) U.S. Cl. 514/473; 514/690; 435/7.32

(57) ABSTRACT

Bacteria lacking the ability to secrete autoinducer-2 may nonetheless be regulated by contacting the bacteria with an amount of an autoinducer-2 effector that is sufficient to regulate the bacterium. Pseudomonas aeruginosa, a bacterium that colonizes the lungs of cystic fibrosis patients with often devastating effects on health, is a preferred target for regulation.

### RELATED APPLICATION INFORMATION

This application claims priority to U.S. Provisional Application No. 60/292,543, filed on May 21, 2001, which is hereby incorporated by reference in its entirety.

### BACKGROUND OF THE INVENTION

1. Field of the Invention

This invention relates generally to methods for regulating bacteria. More particularly, this invention relates to methods of regulating bacteria that do not produce autoinducer-2, by exposing the bacteria to an autoinducer-2 effector.

2. Description of the Related Art

Cystic fibrosis (CF), a genetic disease affecting approximately 30,000 children and adults in the United States, causes the body to produce an abnormally thick, sticky mucus, due to the faulty transport of sodium and chloride (salt) within cells lining organs such as the lungs and pancreas. The thick mucus obstructs the pancreas, preventing enzymes from reaching the intestines to help break down and digest food. In addition, bacteria colonize this mucus and give rise to recurrent infections that lead to chronic inflammation that progressively impairs respiration, eventually resulting in death.

Foremost among these colonizing bacteria is Pseudomonas aeruginosa. Tragically, many cystic fibrosis victims are children. By age 12, 60-90% of cystic fibrosis patients suffer from infection with this bacterium, and most die before age 30. Although other pathogens commonly colonize the respiratory tract of cystic fibrosis patients, Pseudomonas aeruginosa causes almost 90% of the morbidity and mortality of the disease.

Bacteria such as Pseudomonas aeruginosa are pathogens that typically attack host cells in part by producing virulence factors, which are proteins and other compounds that promote development of an infection. Because such virulence factors typically provoke an immune response from the host organism (i.e., they are antigenic), many pathogens delay producing these factors until their population density achieves a level where the bacteria can better withstand an immune system response. Such pathogens monitor their population density by secreting and sensing low molecular-weight compounds, known as autoinducers, a mechanism known as “quorum-sensing.” FIG. 1 schematically illustrates several features of quorum sensing. It is thought that such bacterial quorum sensing systems consist of a secretion/sensing circuit in which individual bacteria secrete the autoinducer, but do not respond to it until the autoinducer concentration reaches a certain threshold that corresponds to the population density of the bacteria.

Three different quorum-sensing systems are now known, with their classification depending on the chemical nature of the autoinducer. For example, Pseudomonas aeruginosa secretes and detects an N-acylhomoserine lactone, an autoinducer also known as AHL, autoinducer-1, or AI-1 (see H. Wu et al., Pseudomonas aeruginosa Mutations in lasl and rhll Quorum Sensing Systems Result in Milder Chronic Lung Infection,” Microbiology, Vol. 147, pp. 1105-1113 (2001)). PCT International Publication No. WO 00/32152 describes a second quorum-sensing system that uses a different type of autoinducer, known as autoinducer-2. WO 00/32152 represents a considerable advance in the art by providing various methods for regulating bacteria. For example, that publication discloses methods for identifying bacteria that have a secretion/sensing quorum sensing circuit and interfering with the functioning of that circuit. In addition, quorum-sensing systems based upon peptides are also known. Those skilled in the art have generally presumed that naturally-occurring bacteria sense only the autoinducer(s) they themselves produce.

In addition to producing virulence factors, often under quorum-sensing control, some bacterial pathogens promote infection in ways thought unconnected with quorum-sensing. One such way is by manipulating the immune response of the host, such as by inducing apoptosis in host immune system cells. Apoptosis, or programmed cell death, a normal process by which the body destroys unneeded, aberrant or diseased cells, activates the immune system to a much lesser extent than does host cell lysis induced by a bacterium. Inducing apoptosis therefore helps the pathogen to escape detection and destruction by the host's immune system. For example, Pseudomonas aeruginosa induces apoptosis of respiratory system cells (see S. Rajan et al., “Pseudomonas aeruginosa Induction of Apoptosis in Respiratory Epithelial Cells,” Am. J. Respir. Cell. Mol. Biol., Vol. 23, pp. 304-312 (2000)), a factor that makes its infections more difficult to overcome.

Reducing this apoptosis would therefore activate the immune system toward the infection and thereby result in a more aggressive immune system response to it. Sometimes, however, the immune system responds too aggressively to an infection, and thereby inadvertently aggravates the host's condition. For example, the immune response to virulence factors and other antigens from an overwhelming bacterial infection may result in widespread inflammation, multi-organ dysfunction and septic shock. In such cases deactivating rather than activating the immune system is needed.

Thus, there is a long-felt need for methods of regulating bacteria, such as methods for regulating the Pseudomonas aeruginosa pathogen that leads to the deaths of many cystic fibrosis patients.

### SUMMARY OF THE INVENTION

The inventors have discovered bacteria that sense autoinducer-2, but do not produce it. This is surprising, because heretofore those skilled in the art generally believed that quorum sensing involved the secretion and detection of a particular autoinducer, and thus that a bacterium unable to produce an autoinducer would also be unable to sense it. The inventors have discovered that this is not the case.

Thus, a preferred embodiment provides a method for regulating a bacterium, comprising: identifying a bacterium that does not produce autoinducer-2, and contacting the bacterium with an amount of an autoinducer-2 effector that is sufficient to regulate the bacterium.

Another preferred embodiment provides a method for treating a subject, comprising: identifying a subject infected with a bacterium that does not produce autoinducer-2; and administering an autoinducer-2 effector to the subject in an amount that is effective to reduce the severity of the infection.

Another preferred embodiment provides a method for treating cystic fibrosis, comprising: identifying a human suffering from cystic fibrosis who is infected with a first bacterium that does not produce autoinducer-2 and a second bacterium that produces a compound having autoinducer-2 activity; and administering an autoinducer-2 antagonist to the subject in an amount that is effective to reduce the severity of the infection caused by the first bacterium.

These and other embodiments are described in greater detail below.

### BRIEF DESCRIPTION OF THE DRAWINGS

These and other aspects of the invention will be readily apparent from the following description and from the appended drawings, which are meant to illustrate and not to limit the invention, and wherein:

FIG. 1 shows how an autoinducer can mediate production of virulence factors in a pathogenic bacterium.

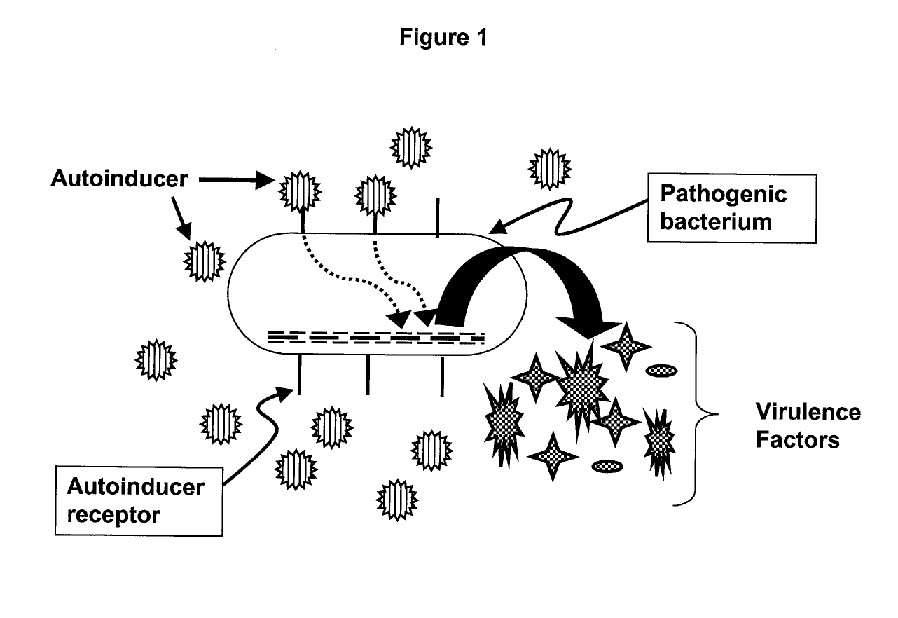

FIG. 2 depicts how AI-2 can influence whether Pseudomonas aeruginosa induces apoptosis or necrosis in a neutrophil.

FIG. 3 is a plot of quantitative pathology data (as a percent of lung tissue showing consolidation) reflecting the immune response observed from rat lung infections with Pseudomonas aeruginosa (designated PAO) under different conditions (case 1: Pseudomonas aeruginosa alone (PAO); case 2: Pseudomonas aeruginosa +AI-2 (PAO/AI-2); case 3: Pseudomonas aeruginosa co-infection with x-hemolytic Streptococcus isolate CFX5 (representing normal respiratory tract flora, designated NF, that secrete AI-2) (PAO/NF); and case 4: Pseudomonas aeruginosa co-infection with α-hemolytic Streptococcus isolate CFX5 (representing normal respiratory tract flora that secrete AI-2) treated with QXO18 (3(2H)-4-hydroxy-5-methylfuranone) (PAO/NF/QXO18)).

FIG. 4 is a microphotograph showing the histopathology of rat lung tissue recovered 7 days after infection with Pseudomonas aeruginosa.

FIG. 5 is a microphotograph showing the histopathology of rat lung tissue recovered 7 days after infection with Pseudomonas aeruginosa in the presence of AI-2.

FIG. 6 is a microphotograph showing the histopathology of rat lung tissue recovered 7 days after infection with Pseudomonas aeruginosa and α-hemolytic Streptococcus isolate CFX5 (representing normal respiratory tract flora that secrete AI-2).

FIG. 7 is a microphotograph showing the histopathology of rat lung tissue recovered 7 days after infection with Pseudomonas aeruginosa and α-hemolytic Streptococcus isolate CFX5 (representing normal respiratory tract flora that secrete AI-2) after five days of treatment with an aerosol of 3(2H)-4-hydroxy-5-methylfuranone.

### DETAILED DESCRIPTION OF THE PREFERRED EMBODIMENT

In preferred embodiments, this invention involves regulating a bacterium. In this context, “regulating” means controlling one or more functions of the bacterium. Examples of such bacteria functions include the production of virulence factors and the induction of apoptosis in the cells of the host. Such regulating may take place in vitro or in vivo, and may involve enhancing or diminishing one or more bacterial functions.

In preferred embodiments, this invention involves identifying a bacterium that does not produce autoinducer-2. Autoinducer-2 is the non-homoserine lactone autoinducer produced by V. harveyi. A recent report indicates that autoinducer-2 has the structure

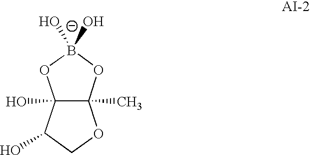

(see X. Chen, S. Schauder, N. Potier, A. Van Dorsselaer, I. Pelczer, B. Bassler, and F. Hughson, Nature, Vol. 415, pp. 545-549 (Jan. 31, 2002)). In a preferred embodiment, the bacterium does not produce a compound having autoinducer-2 activity. Autoinducer-2 is an example of a compound having autoinducer-2 activity. Other examples of compounds having autoinducer-2 activity include 3(2H)-furanones such as 3(2H)-4-hydroxy-5-methylfuranone. Methods for identifying a bacterium that does not produce autoinducer-2 are known to those skilled in the art and include examination of bacterial genome databases for the absence of the luxS gene in a specific genome, Southern hybridization of genomic DNA using a labeled luxS gene probe or using the luxS specific DNA primers in a polymerase chain reaction (PCR) to determine the presence/absence of luxS (see methods described in WO 00/32152 and WO 01/85664, both of which are hereby incorporated by reference). A preferred method for identifying a bacterium that does not produce autoinducer-2 is conducted by using a bioassay that directly measures the activity of autoinducer-2 in cell-free culture supernatants of the bacterium in question. This approach employs the use of a bioluminescent bacterium, such as Vibrio harveyi strain BB170, that is modified to emit light only in the presence of autoinducer-2. When exposed to cell-free culture supernatants of bacteria that produce autoinducer-2, strain BB170 will emit light in direct proportion to the concentration of autoinducer-2 in the sample. Culture supernatants from bacteria that do not produce autoinducer-2 will not elicit significant light production from BB170. Examples of bacteria that do not produce autoinducer-2 include bacteria from the genus Pseudomonas, e.g., Pseudomonas aeruginosa, and bacteria from the genus Burkholderia, e.g., Burkholderia cepacia.

Surprisingly, we have discovered that bacteria that do not produce autoinducer-2 can be regulated by contacting the bacteria with an autoinducer-2 effector. Pseudomonas aeruginosa is an example of such a bacterium. This invention is not bound by theory, but it is believed that these bacteria have the ability to sense and/or respond to autoinducer-2, but lack the ability to secrete it. In essence, these bacteria lack the secretion half of the usual quorum sensing circuit, and only possess the sensing half. To the extent that the pathogenic effects of naturally occurring bacteria are affected by autoinducer-2 or a compound having autoinducer-2 activity, this invention provides a way for controlling those pathogenic effects by contacting such bacteria with an autoinducer-2 effector. Bacteria that do not produce autoinducer-2 but respond to an autoinducer-2 effector may be referred to herein as “target” bacteria.

In this context an “autoinducer-2 effector” is an autoinducer-2 agonist or antagonist, that is, a compound having autoinducer-2 activity in some bacterium, or blocking such activity by autoinducer-2 itself, respectively.

Target bacteria have receptors that sense autoinducer-2 and thus enable them to respond to autoinducer-2 by performing various pathogenic bacterial functions. It is believed that an autoinducer-2 antagonist functions by inhibiting detection of autoinducer-2 (e.g., AI-2 synthesized by a second bacterium in the presence of the target bacterium) or by blocking such receptors, and thus inhibiting the resultant pathogenic effects. It is also believed that an autoinducer-2 agonist functions like autoinducer-2 by stimulating production of virulence factors, doing so by binding to the receptor in a manner like autoinducer-2.

Methods for identifying autoinducer-2 effectors include measuring how a compound affects the expression of one or more proteins whose expression autoinducer-2 regulates (see methods described in WO 00/32152 and WO 01/85664, both of which are hereby incorporated by reference). Autoinducer-2 agonists increase the level of expression of such proteins, while autoinducer-2 antagonists decrease it. A preferred method for identifying an autoinducer-2 effector involves measuring the activity of a reporter protein or other species whose expression is regulated by autoinducer-2. Examples of such systems are green fluorescent protein (GFP) or luciferase (lux) reporter systems. In such a system, the gene(s) encoding the reporter are cloned in-frame and downstream of a gene regulated by autoinducer-2. The expression of the gene induced by autoinducer-2 is then measured by quantifying the resulting fluorescence (GFP) or bioluminescence (lux).

Preferred examples of bacterial functions that can be suppressed by an autoinducer-2 antagonist include production of virulence factors, apoptosis of host cells, and, more preferably, apoptosis of host immune system cells. Preferred autoinducer-2 antagonists comprise a cyclopentenone group, e.g., 2-alkyl-2-cyclopenten-1-ones having from about 6 to about 14 carbons, as in formula (I) below. In formula (I), n is preferably an integer in the range of 3 to 5. An example of a preferred 2-alkyl-2-cyclopenten-1-one is 2-pentyl-2-cyclopenten-1-one, wherein n=4.

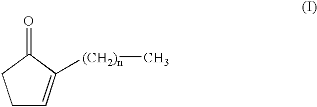

The autoinducer-2 effector may be an autoinducer-2 agonist. A preferred autoinducer-2 agonist initiates or enhances a bacterial function normally initiated by the presence of autoinducer-2. Preferred examples of such functions include therapeutic apoptosis of host cells, more preferably therapeutic apoptosis of host immune system cells. Examples of autoinducer-2 agonists include 3(2H)-furanones such as 3(2H)-4-hydroxy-5-methylfuranone, 3(2H)-4-hydroxy 2,5-methylfuranone, and 3(2H)-4-methoxy-2,5-methylfuranone.

A target bacterium is preferably regulated by exposing the bacterium to an amount of an autoinducer-2 effector that is sufficient to regulate the bacterium. Such amounts can be determined by identifying a bacterial function of interest, exposing the bacterium to various amounts of the autoinducer-2 effector under controlled conditions, and measuring the change in the bacterial function of interest as a function of the amount of autoinducer-2 effector. Exposing the bacterium to the autoinducer-2 effector may be carried out in vitro or in vivo. Amounts of autoinducer-2 effector sufficient to regulate the bacterium may vary depending on the conditions under which the bacterial function is measured. For example, a bacterium may be in the presence of a pre-existing amount of autoinducer-2, an autoinducer-2 agonist, and/or an autoinducer-2 antagonist, such that the amount of added autoinducer-2 effector that is sufficient to regulate the bacterium changes.

The autoinducer-2 effector may be formulated by combining it with other substances, depending on the desired method for contacting it with the target bacterium. Preferably, a pharmaceutically acceptable composition comprises the autoinducer-2 effector as described hereinbelow. It is understood that the description herein of various ways of contacting the autoinducer-2 effector with a target bacterium as described herein also apply to pharmaceutical compositions comprising an autoinducer-2 effector.

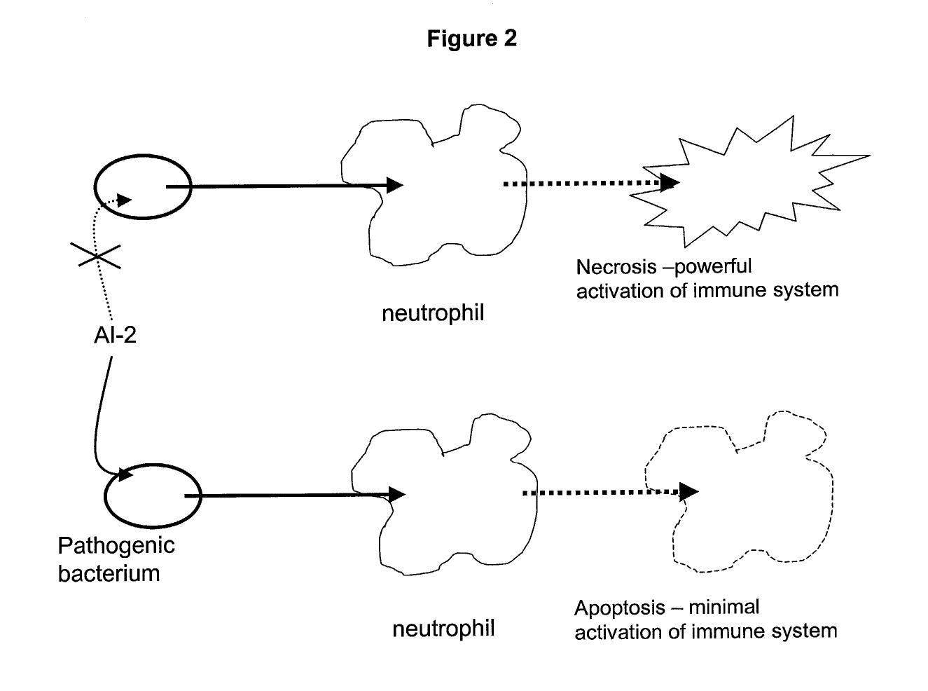

The target bacterium may induce apoptosis of host cells. Such apoptosis may be either a host defense mechanism or reflect the pathogenesis of the infection, and thus may be advantageous or disadvantageous to the host. See M. Behnia et al., “Lung Infections—Role of Apoptosis in Host Defense and Pathogenesis of Disease, Chest, Vol. 117 (6), pp. 1771-1777 (2000). For example, it is believed that apoptosis of the host defense cells favors the host when the pathogen exists within the host macrophages, whereas, for extracellular infections, apoptosis of host inflammatory cells favors the pathogen. Also, in some cases (such as septic shock) it may be desirable to suppress the response of the immune system, in which case target bacteria are preferably regulated so as to increase apoptosis of eukaryotic cells, preferably immune system cells. Thus, depending on the type of target bacterium and condition of the patient (for in vivo applications), effective regulation by contacting with an autoinducer-2 effector may involve increasing or decreasing the extent to which the target bacterium induces apoptosis of host cells (see FIG. 2).

In one embodiment, the bacterium is in the presence of autoinducer-2 or an autoinducer-2 agonist. The autoinducer-2 or an autoinducer-2 agonist may have been added deliberately or may have been secreted by a second bacterium. For example, in cystic fibrosis, the lungs may be infected by both Pseudomonas aeruginosa and a second bacterium that secretes autoinducer-2 or an autoinducer-2 agonist. Examples of such second bacteria include α-hemolytic Streptococcus, Staphylococcus aureus, M. tuberculosis, Haemophilus influenzae, and S. pneumoniae. This invention is not bound by theory, but it is believed that the pathogenic activity of the target bacterium (which responds to but does not secrete autoinducer-2) may be initiated or enhanced by the second bacterium. Without effective regulation, the second bacterium secretes autoinducer-2 and/or a compound having autoinducer-2 activity that is sensed by the target bacterium, causing it to produce virulence factors, induce host cell apoptosis, etc. These pathogenic activities can be regulated by contacting the target bacterium with an autoinducer-2 effector, preferably with an amount of an autoinducer-2 antagonist that is sufficient to suppress or prevent the pathogenic activities.

The target bacterium is often in the presence of eukaryotic cells. For example, in cystic fibrosis, the lungs are often infected by Pseudomonas aeruginosa, and thus the target bacterium is in the presence of numerous types of eukaryotic cells, including those from the respiratory and immune system. Pseudomonas aeruginosa produces virulence factors and can induce apoptosis of respiratory system cells, and it is further believed that it can induce apoptosis of immune system cells. Pseudomonas aeruginosa lung infections in cystic fibrosis patients are particularly devastating because the bacterium not only produces virulence factors, but also suppresses the ability of the immune system to respond effectively.

In a preferred embodiment, a target bacterium that has two or more pathogenic activities, e.g., production of virulence factors and apoptosis of eukaryotic cells, is regulated by contacting the bacterium with a autoinducer-2 effector that diminishes the effects of two or more of the pathogenic activities. This embodiment is particularly advantageous when the target bacterium infects a host because the host not only benefits from a reduction in virulence but also by maintaining a competent immune system with which to eliminate the bacterium. A further benefit is that this embodiment often results in the application of far milder selective pressure to the bacterium by targeting a non-essential signaling mechanism instead of a cellular target, decreasing the likelihood that the bacterium will develop resistance mechanisms.

A preferred embodiment provides a method for treating a subject infected by one or more target bacteria and, optionally, one or more secondary bacteria. This method preferably comprises first identifying a subject infected with a target bacterium. Such subjects may be identified in a number of ways. Typically, cystic fibrosis patients are chronically infected with Pseudomonas aeruginosa and a mixed population of AI-2 producing bacteria called “normal flora”. Present anti-bacterial treatments for cystic fibrosis patients presume the presence of Pseudomonas (or closely related Burkholderia cepacia). Classical microbiological methods, such as plating and colony characterization or microscopy, can be used to confirm the presence of target bacteria. Such methods are described in standard textbooks (see “Clinical and Pathogenic Microbiology”; Howard, B. J., Keiser, J. F., Smith, T. F., Weissfeld, A. S., Tilton, RC. (eds.) Mosby-Year Book, Inc., St. Louis, Mo., 2nd edition (January 1994)).

Preferably, the identified subject is a human. A preferred method involves treating a human suffering from cystic fibrosis or septic shock. Methods for identifying humans suffering from cystic fibrosis or septic shock are known to those skilled in the art. A more preferred method involves treating a human infected with a target bacterium belonging to a genus selected from the group consisting of Pseudomonas and Burkholderia, most preferably Pseudomonas aeruginosa or Burkholderia cepacia. Such bacterial infections may be identified through known culture techniques. A particularly preferred method involves treating a patient suffering from cystic fibrosis who is infected with a first bacterium that does not produce autoinducer-2 and a second bacterium that produces autoinducer-2 or a compound having autoinducer-2 activity. Methods for identifying target bacteria are described above, and the second bacteria are identified as those that are not target bacteria.

The autoinducer-2 effectors disclosed herein are preferably administered to subjects in the form of pharmaceutical compositions comprising the autoinducer-2 effector. For example, a preferred mode of administration of the autoinducer-2 effector is via inhaled aerosol containing autoinducer-2 effector. U.S. Pat. No. 5,508,269, the disclosure of which is hereby incorporated by reference in its entirety, describes methods of administering therapeutic agents to the lungs through inhalation of an aerosol, with particular reference to treating cystic fibrosis patients.

Another preferred mode of administration of the autoinducer-2 effector is oral. Oral compositions preferably include an inert diluent and/or an edible carrier. The autoinducer-2 effector can be enclosed in gelatin capsules or compressed into tablets. For the purpose of oral therapeutic administration, the autoinducer-2 effector can be incorporated with excipients and used in the form of tablets, troches, or capsules. Pharmaceutically compatible binding agents and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; and/or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents. The compound can be administered as a component of an elixir, suspension, syrup, wafer, chewing gum or the like. A syrup may contain, in addition to the active compounds, sucrose as a sweetening agent and certain preservatives, dyes and colorings and flavors.

The autoinducer-2 effector can also be mixed with other active materials that do not impair the desired action, or with materials that supplement the desired action, such as antibiotics. Preferred antibiotics for this purpose include aminoglycosides such as tobramycin, glycopeptides such as vancomycin, beta lactams such as amoxicillin, quinolones such as ciprofloxicin, macrolides such as azithromycin, tetracyclines, sulfonamides, trimethoprim-sulfamethoxazole, or chloramphenicol. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; chelating agents such as ethylenediaminetetraacetic acid (EDTA); buffers such as acetates, citrates or phosphates and agents for the adjustment of tonicity such as sodium chloride or dextrose. The parental preparation can be enclosed in ampoules, disposable syringes or multiple dose vials made of glass or plastic. If administered intravenously, preferred carriers are physiological saline or phosphate buffered saline (PBS).

In a preferred embodiment, the autoinducer-2 effector is prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are known to those skilled in the art.

Liposomal suspensions (including liposomes targeted with monoclonal antibodies to surface antigens of specific cells) are also pharmaceutically acceptable carriers. These may be prepared according to methods known to those skilled in the art, for example, as described in U.S. Pat. No. 4,522,811, which is hereby incorporated by reference in its entirety. For example, liposome formulations may be prepared by dissolving appropriate lipid(s) (such as stearoyl phosphatidyl ethanolamine, stearoyl phosphatidyl choline, arachadoyl phosphatidyl choline, and/or cholesterol) in an inorganic solvent that is then evaporated, leaving behind a thin film of dried lipid on the surface of the container. An aqueous solution of the autoinducer-2 effector is then introduced into the container. The container is then swirled by hand to free lipid material from the sides of the container and to disperse lipid aggregates, thereby forming the liposomal suspension.

For parenteral administration, the active compound is preferably formulated in a unit dosage injectable form (solution, suspension, emulsion) in association with a pharmaceutically acceptable parenteral vehicle. Such vehicles are preferably non-toxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. Autoinducer-2 effectors are preferably formulated in such vehicles at concentrations of about 10 nanograms/ml to about 100 milligrams/ml, more preferably about 10 micrograms/ml to about 10 milligrams/ml.

The autoinducer-2 effectors disclosed herein (including pharmaceutical compositions comprising these compounds) are preferably administered to subjects in therapeutically effective amounts. A therapeutically effective amount is an amount that is effective to decrease the pathogenic effects of the target bacterium, e.g., to reduce the severity of the infection caused by the first bacterium and/or to ease the symptoms of the condition from which the subject is suffering. Preferred therapeutically effective amounts can vary over a broad range. The dose and dosage regimen is preferably selected by considering the nature of the subject's need for regulation of the target bacterium, the characteristics of the particular autoinducer-2 effector, e.g., its therapeutic index, the subject, the subject's history and other factors known to those skilled in the art. Preferred daily dosages of autoinducer-2 effector are typically in the range of about 1 microgram/kg to about 1,000 milligrams/kg of subject weight, although higher or lower doses may be used in appropriate circumstances. More preferably, daily dosages of autoinducer-2 effector are typically in the range of about 10 micrograms/kg to about 10 milligrams/kg of subject weight, or an equivalent sustained release dosage.

Therapeutically effective amounts can be determined by those skilled in the art by such methods as clinical trials. Dosage may be adjusted in individual cases as required to achieve the desired degree of target bacterial regulation. Sustained release dosages and infusions are specifically contemplated. Active compounds can be administered by any appropriate route for systemic, local or topical delivery, for example, orally, parenterally, intravenously, intradermally, subcutaneously, buccally, intranasally, by inhalation, vaginally, rectally or topically, in liquid or solid form. Methods of administering the compounds described herein may be by specific dose or by controlled release vehicles. Inhalation is preferred for the treatment of lung infections such as those experienced in cystic fibrosis.

The autoinducer-2 effector may be administered at once, or may be divided into a number of smaller doses to be administered at varying intervals of time. It is to be further understood that for any particular subject, specific dosage regimens should be adjusted over time according to the individual need and the professional judgment of the person administering or supervising the administration of the autoinducer-2 effector, and that the concentration ranges set forth herein are exemplary only and are not intended to limit the scope or practice of the claimed methods.

### EXAMPLES

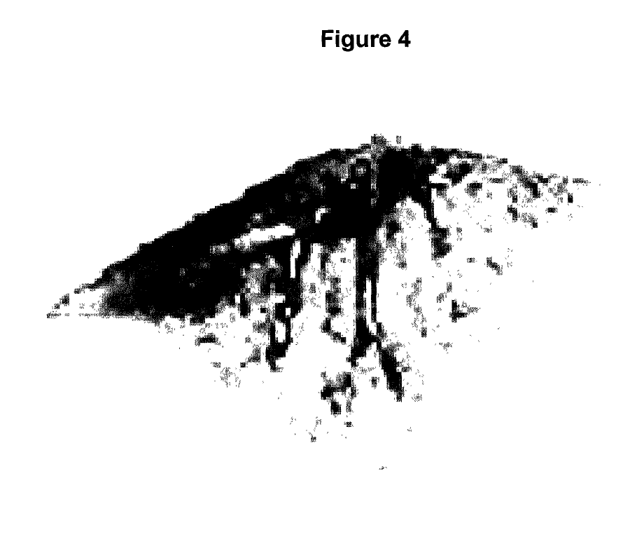

The following Examples illustrate the effect of normal flora and the presence of autoinducer-2 (AI-2) on Pseudomonas aeruginosa infection in the agar bead model of rat chronic respiratory infection. Groups of 15 rats (male Sprague-Dawley 180-200 grams) were inoculated intratracheally with 0.05 mL suspensions of bacteria embedded in agar beads. Seven days post-infection each group was treated as follows: (1) five rats—lungs removed for quantitative microbiology; (2) five rats—bronchoaveolar lavage (BAL) were obtained and analyzed for total cell counts, neutrophils, neutrophil elastase and AI-2 activity; and (3) five rats—lungs removed for histology (quantitative pathology). In the microphotographs shown in FIGS. 4-7, the dark areas indicate regions of consolidation, an indication of tissue damage. An α-hemolytic Streptococcus isolate (designated CFX5) from a cystic fibrosis patient was used to supply naturally synthesized AI-2 in vivo.

### EXAMPLE 1

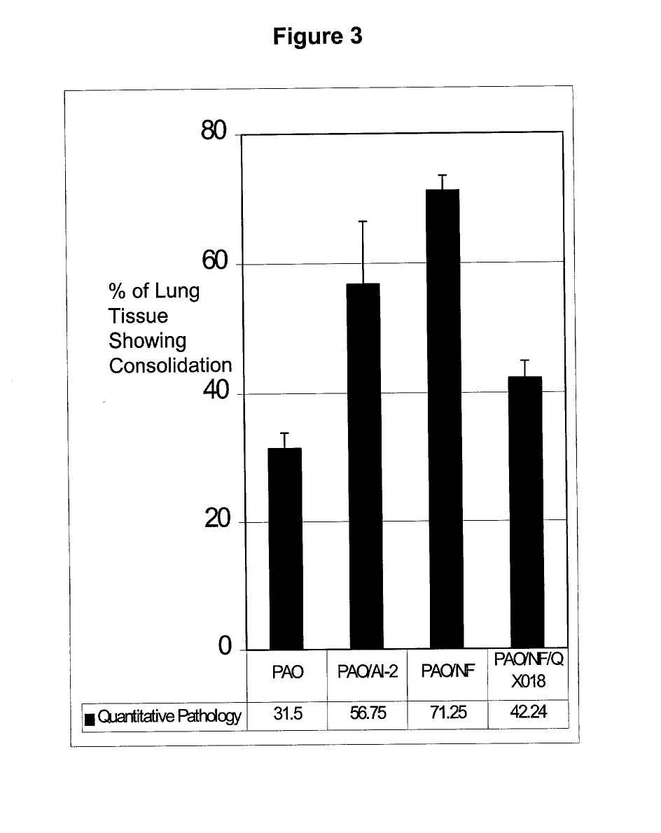

Rats inoculated with agar beads containing Pseudomonas aeruginosa alone yielded the quantitative pathology shown in FIG. 3 (PAO), and the histopathology shown in the microphotograph of FIG. 4

### EXAMPLE 2

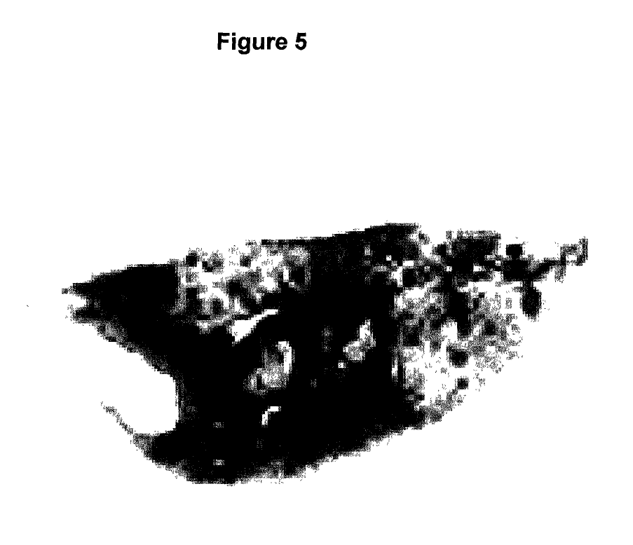

Rats inoculated with agar beads containing Pseudomonas aeruginosa alone and exposed to synthetic AI-2 yielded the quantitative pathology shown in FIG. 3 (PAO/AI-2), and the histopathology shown in FIG. 5.

### EXAMPLE 3

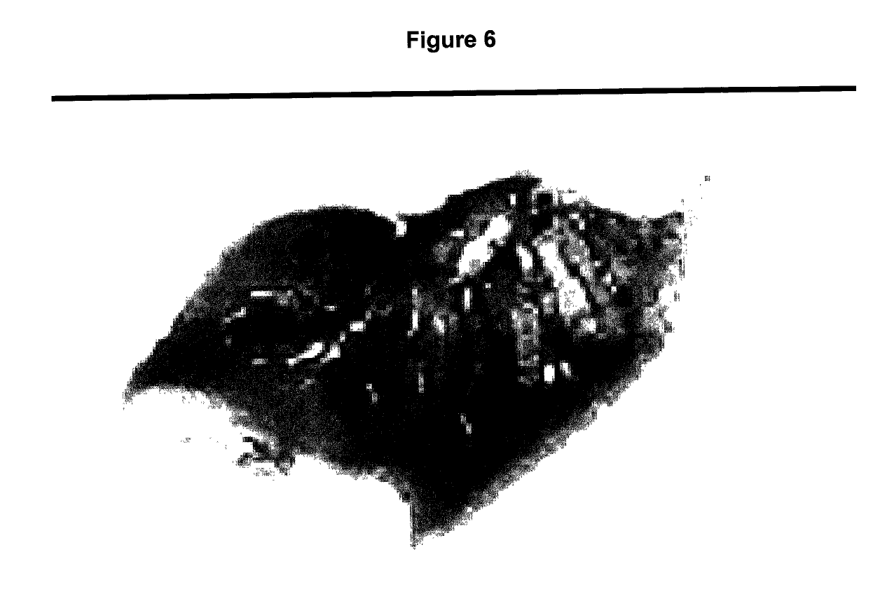

Rats inoculated with agar beads containing Pseudomonas aeruginosa +α-hemolytic Streptococcus (CFX5) yielded the quantitative pathology shown in FIG. 3 (PAO/NF), and the histopathology shown in FIG. 6.

### EXAMPLE 4

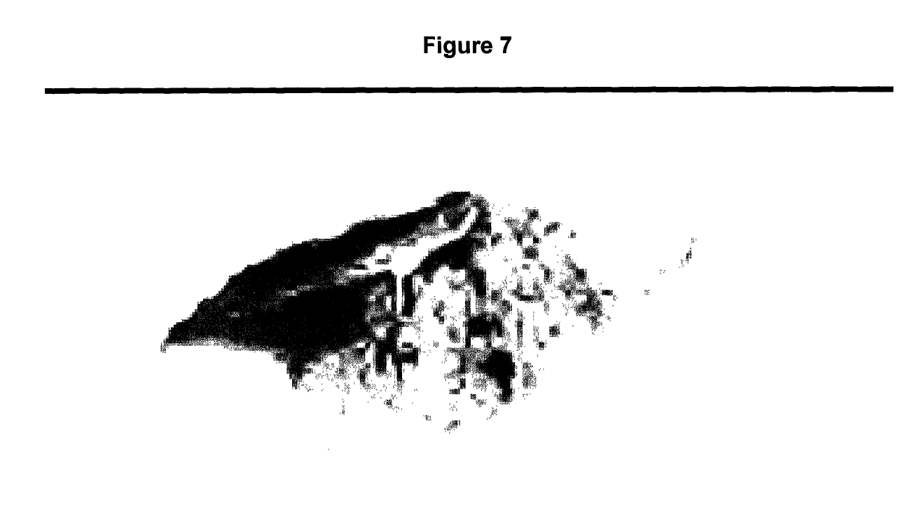

Rats inoculated with agar beads containing Pseudomonas aeruginosa +α-hemolytic Streptococcus (CFX5), treated daily for six days with aerosolized QXO18 (2-pentyl-2-cyclopenten-1-one), an AI-2 antagonist, yielded the quantitative pathology shown in FIG. 3 (PAO/NF/QXO18), and the histopathology shown in FIG. 7.

The results obtained in Examples 1-4 (summarized in FIG. 3) show that AI-2 increases the lung pathology caused by Pseudomonas aeruginosa, and that an AI-2 antagonist greatly diminishes this increase. Specifically, comparison of Examples 1 (“PAO”) and 2 (“PAO/AI-2”) shows that exogenously supplied AI-2 increases the lung pathology caused by Pseudomonas aeruginosa. Example 3 (“PAO/NF”) shows that AI-2 (supplied endogenously by an exemplary normal flora, α-hemolytic Streptococcus isolate (CFX5) that secretes Al-2) increases the lung pathology even more. Example 4 establishes that the AI-2 antagonist 2-pentyl-2-cyclopenten-1-one (“PAO/NF/QX018”) greatly diminishes the increased lung pathology arising from endogenously supplied AI-2.

It will be appreciated by those skilled in the art that various omissions, additions and modifications may be made to the processes described above without departing from the scope of the invention, and all such modifications and changes are intended to fall within the scope of the invention, as defined by the appended claims.

What is claimed is:

1. A method for regulating a bacterium, comprising:
identifying a bacterium that does not produce autoinducer-2; and
contacting the bacterium with an amount of an autoinducer-2 effector that is sufficient to regulate the bacterium.

2. The method of claim 1 in which the bacterium belongs to a genus selected from the group consisting of Pseudomonas and Burkholderia.

3. The method of claim 2 in which the bacterium is Pseudomonas aeruginosa or Burkholderia cepacia.

4. The method of claim 1 in which the bacterium is in the presence of autoinducer-2 or an autoinducer-2 agonist.

5. The method of claim 4 in which the autoinducer-2 or autoinducer-2 agonist is secreted by a second bacterium.

6. The method of claim 4 in which the bacterium senses the autoinducer-2 or autoinducer-2 agonist.

7. The method of claim 6 in which the autoinducer-2 or autoinducer-2 agonist comprises a furanone.

8. The method of claim 7 in which the furanone is 3(2H)-4-hydroxy-5-methylfuranone.

9. The method of claim 1 in which the bacterium is in the presence of eukaryotic cells.

10. The method of claim 9 in which the eukaryotic cells are immune system cells.

11. The method of claim 10 in which the bacterium causes apoptosis of the immune system cells.

12. The method of claim 1 in which the autoinducer-2 effector is an autoinducer-2 antagonist.

13. The method of claim 12 in which the autoinducer-2 antagonist comprises a cyclopentenone group.

14. The method of claim 13 in which the autoinducer-2 antagonist is a 2-alkyl-2-cyclopenten-1-one having from about 6 to about 14 carbons.

15. The method of claim 14 in which the autoinducer-2 antagonist is 2-pentyl-2-cyclopenten-1-one.

16. The method of claim 1 in which the autoinducer-2 effector is an autoinducer-2 agonist.

17. A method for treating a subject, comprising:
identifying a subject infected with a bacterium that does not produce autoinducer-2; and
administering an autoinducer-2 effector to the subject in an amount that is effective to reduce the severity of the infection.

18. The method of claim 17 in which the subject is a human.

19. The method of claim 18 in which the subject is suffering from cystic fibrosis.

20. The method of claim 18 in which the subject is suffering from septic shock.

21. The method of claim 19 in which the bacterium belongs to a genus selected from the group consisting of Pseudomonas and Burkholderia.

22. The method of claim 21 in which the bacterium is Pseudomonas aeruginosa or Burkholderia cepacia.

23. A method for treating cystic fibrosis, comprising:
identifying a human suffering from cystic fibrosis who is infected with a first bacterium that does not produce autoinducer-2 and a second bacterium that produces a compound having autoinducer-2 activity; and
administering an autoinducer-2 antagonist to the subject in an amount that is effective to reduce the severity of the infection caused by the first bacterium.

24. The method of claim 23 in which the first bacterium is Pseudomonas aeruginosa.

25. The method of claim 23 in which the compound having autoinducer-2 activity is autoinducer-2.
